(19) United States
(12) Patent Application Publication
(10) Pub. No.: US 2020/0316141 A1
(43) Pub. Date: Oct. 8, 2020

(54) Composition To Support Healthy Brain Function

(71) Applicant: The Procter & Gamble Company, Cincinnati, OH (US)

(72) Inventors: Arvind Venkataraman, Mason, OH (US); Chelsay Lynn Brewster, Hamilton, OH (US); Kimberly Conner Kozak, Lebanon, OH (US); Lijuan Li, Lebanon, OH (US)

(21) Appl. No.: 16/838,046

(22) Filed: Apr. 2, 2020

Related U.S. Application Data
(60) Provisional application No. 62/827,994, filed on Apr. 2, 2019.

(51) Int. Cl. A61K 35/742; A61K 35/745; A61K 35/747; A61K 36/06; A61K 38/43

(52) U.S. Cl. CPC A61K 35/742; A61K 35/745; A61K 38/43; A61K 36/06; A61K 35/747

(57) ABSTRACT

Described herein is a composition containing probiotic bacteria and a fermentate that can increase IPA production. The composition comprises one or more bacteria having a nucleic acid sequence with at least 80% homology to the nucleic acid sequence of SEQ ID NO: 1, a fermentate comprising soy flour, a yeast, and a proteolytic enzyme, and an excipient, carrier, and/or diluent. The composition can promote brain health and/or nervous system function.

### FIELD

Described herein is a composition that can support healthy brain and/or nervous system function comprising a combination of a bacteria and a fermentate that can increase indole-3-propionic acid production.

### INCORPORATION BY REFERENCE OF THE SEQUENCE LISTING

This application contains, as a separate part of disclosure, a Sequence Listing in computer-readable form (filename: 15496P_ST25.txt; Size: 7,110 bytes; Created: Apr. 2, 2019) which is incorporated by reference herein in its entirety.

### BACKGROUND

Practicing good nutrition can be challenging. Some people seek supplements to provide additional nutrients to improve their health and wellness, including maintaining healthy brain function. The brain is particularly susceptible to oxidative stress due to its high rate of oxygen consumption, its large content of polyunsaturated fatty acids and regional high iron levels, and its proportionately low antioxidant capacity. It is known that oxidative stress can cause reduced neurogenesis and increased neuronal death. It has been shown that cognitive impairment is related to oxidative stress and an efficient antioxidant system can preserve the cognitive function in older adults.

Indole-3-propionic acid (“IPA”) is a neuroprotective antioxidant that may improve mood, cognition, and/or maintain healthy brain function and nervous system in humans. IPA is made by the gut microbiome in the colon and crosses the intestinal epithelium and blood brain barrier to enter the brain. In the brain, IPA has been shown to play a protective role as an antioxidant, thereby protecting the structure & function of neurons. It is believed that the antioxidant property of IPA can play a key role in promoting brain health. It is also well known that the consumption of IPA by mouth can increase IPA levels in situ. (See Kaufmann SHE. 2018. Indole propionic acid: a small molecule links between gut microbiota and tuberculosis. Antimicrob Agents Chemother 62:e00389-18; Niebler G. NCT01898884: Safety and Pharmacology Study of VP 20629 in Adults With Friedreich's Ataxia (2018).

Despite the growing appreciation of the beneficial effects of IPA on brain health, currently IPA is only commercially produced in a chemically synthesized form. However, an increasing number of consumers have an interest in understanding product ingredients, including their origin, and prefer supplements from natural sources. The direct ingestion of chemically synthesized IPA may not be preferred by these natural-seeking consumers. Furthermore, along with IPA, other indole derivatives such as indole-3-acetic acid, indole-3-acrylic acid, and indole-3-lactic acid are also emerging as providing positive health benefits. However, chemically synthesized forms of IPA only deliver pure IPA.

Thus, there is a need for a naturally derived means of providing a combination of indole derivatives, within which IPA would be a major component, in order to promote brain health.

### SUMMARY

Described herein is a composition comprising: (a) one or more bacteria having a nucleic acid sequence with at least 80% homology to the nucleic acid sequence of SEQ ID NO: 1; (b) a fermentate comprising soy flour, a yeast, and a proteolytic enzyme; and (c) an excipient, carrier, and/or diluent.

Described herein is a composition comprising: (a) one or more bacteria having a nucleic acid sequence with at least 80% homology to the nucleic acid sequence of SEQ ID NO: 1; (b) a fermentate comprising a yeast; and (c) an excipient, carrier, and/or diluent; wherein the one or more bacteria produce from about 5 to about 80 μg/mL of indole-3-propionic acid (IPA) after 24 hours of anaerobic in vitro incubation at 36° C. with the fermentate.

### DETAILED DESCRIPTION

Consumers are looking for effective and natural ways of supplementing their diets with IPA in order to promote brain and mental well-being. Described herein is a composition comprising one or more bacteria that can produce increased levels of IPA and other indole derivatives when combined with a fermentate. It has been surprisingly found that when certain fermentates are added to bacteria, IPA production can significantly increase. In particular, it was found that a fermentate comprising yeast could significantly increase the production of IPA by Clostridium sporogenes. In some aspects, the fermentate can comprise yeast, soy flour, and a proteolytic enzyme.

As used herein, the terms “administer,” “administering,” and “administration,” refer to any method which, in sound medical practice, delivers the composition to a subject in such a manner as to provide a therapeutic effect.

As used herein, “anaerobic conditions” refer to any growth or nutrient conditions that exclude the presence of oxygen (e.g., less than about 1 ppm free oxygen, preferably less than about 0.1 ppm free oxygen, more preferably from about 0 to about 1 ppm free oxygen).

As used herein, the abbreviation “CFU” (“colony forming units”) designates the number of bacterial cells revealed by microbiological counts on agar plates, as will be commonly understood in the art.

As used herein, “fermentation” refers to a process by which microorganisms metabolize raw materials.

As used herein, “fermentate” refers to the isolated solids after removal of water from a fermentation medium.

The terms “microbes” and “microorganisms” are used interchangeably herein to refer to bacteria. The terms “microbiome”, “microbiota”, and “microbial habitat” are used interchangeably herein and can refer to the ecological community of microorganisms that live on or in a subject's body. Microbiomes can exist on or in many, if not most parts of the subject. Some non-limiting examples of habitats of microbiome can include: body surfaces, body cavities, body fluids, the gut, the colon, skin surfaces and pores, vaginal cavity, umbilical regions, conjunctival regions, intestinal regions, the stomach, the nasal cavities and passages, the gastrointestinal tract, the urogenital tracts, saliva, mucus, and feces.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded and represent the sense or antisense strand. The nucleic acid sequence can be made up of adenine, guanine, cytosine, thymine, and uracil (A, T, C, G, and U) as well as modified versions (e.g. N6-methyladenosine, 5-methylcytosine, etc.).

The terms “subject” refers to any animal subject, including humans, laboratory animals, livestock, and household pets.

As used herein, the articles “a” and “an” are understood to mean one or more of the material that is claimed or described, for example, “an active ingredient” or “a probiotic”.

The composition can contain, consist of, or consist essentially of, the essential elements and limitations of the invention described herein, as well as any additional or optional ingredients, components, or limitations described herein or otherwise useful in compositions intended for use or consumption by a subject.

In various aspects, the composition comprises one or more strains or species of bacteria, a fermentate comprising yeast, and a physiologically, pharmaceutically, or nutritionally acceptable excipient, carrier and/or diluent.

The composition can comprise one or more bacteria that can produce IPA and/or indole derivates such as indole-3-acetic acid, indole-3-acrylic acid, and indole-3-lactic. In some aspects, the composition can comprise one or more bacteria having a nucleic acid sequence that is substantially homologous to the nucleic acid sequence of SEQ ID NO: 1 (Table 1), which encodes the phenyllactate dehydratase gene clusters (fldL, fldI, and fldABC).

TABLE 1
DNA sequence

| SEQ | |
|---|---|
| ID | |
| NO | Nucleotide Sequence |
| 1 | AATTCCCTTTAACAGATACAGGTAAAATTAAGAGGCATGAACTA |
| | AAAAAATGCTTTGAAAAGAAGTTTGAATTAAGACAATCTATTTA |
| | AATTAATAATAAATATATTAAATTAACAATAAATATATTAAATT |
| | AACAATAAATCTATTTAAGGAGGCTTTTTTTATGGAAAACAATA |
| | CAAATATGTTTAGTGGAGTAAAGGTTATTGAATTAGCAAATTTT |
| | ATAGCTGCTCCAGCAGCAGGTAGATTTTTTGCTGATGGTGGTGC |
| | AGAGGTAATAAAAATTGAATCACCTGCTGGAGATCCTTTAAGAT |
| | ATACTGCTCCTTCAGAAGGAAGACCATTAAGCCAAGAAGAAAAT |
| | ACTACTTATGATTTGGAAAATGCAAATAAAAAAGCAATAGTATT |
| | AAATCTTAAAAGCGAAAAAGGTAAAAAGATATTACATGAAATGT |
| | TAGCAGAAGCAGATATATTATTAACTAATTGGAGAACAAAGGCT |
| | TTAGTTAAACAAGGATTAGACTATGAAACACTAAAAGAAAAATA |
| | TCCTAAATTAGTTTTTGCACAAATAACTGGTTATGGTGAAAAAG |
| | GACCAGATAAAGATCTTCCAGGCTTTGATTATACTGCATTTTTC |
| | GCTAGAGGCGGTGTTTCAGGTACTCTTTATGAAAAAGGAACTGT |
| | GCCTCCAAATGTTGTTCCAGGACTTGGAGACCATCAAGCTGGGA |
| | TGTTTTTAGCAGCGGGTATGGCAGGAGCTTTATATAAAGCAAAA |
| | ACAACAGGACAAGGAGATAAAGTAACAGTAAGTTTAATGCATAG |
| | TGCTATGTATGGACTAGGTATTATGATACAAGCTGCTCAATATA |
| | AAGATCATGGATTAGTATATCCGATAAATCGTAATGAAACTCCA |
| | AATCCTTTTATAGTTTCATATAAATCTAAGGATGATTACTTTGT |
| | TCAAGTATGTATGCCACCATATGATGTTTTCTATGATAGATTTA |
| | TGACCGCTTTAGGAAGAGAAGATTTAGTTGGAGACGAAAGATAC |
| | AATAAAATAGAAAATTTAAAAGATGGACGTGCTAAGGAAGTATA |
| | CAGTATAATCGAACAACAAATGGTTACAAAGACAAAGGATGAAT |
| | GGGATAACATATTTAGAGATGCAGACATTCCATTTGCTATCGCA |
| | CAAACTTGGGAAGATTTATTAGAAGATGAACAAGCTTGGGCAAA |
| | TGATTATTTGTATAAGATGAAATATCCAACAGGAAACGAAAGAG |
| | CATTAGTAAGACTTCCAGTATTCTTTAAAGAAGCAGGATTACCA |
| | GAATATAATCAATCACCACAAATAGCAGAAAATACTGTAGAAGT |
| | TTTAAAAGAAATGGGATATACAGAACAAGAGATTGAGGAATTAG |
| | AAAAAGATAAAGATATAATGGTAAGGAAGGAAAAATAATGGCAG |
| | ACATTTATACTATGGGTGTAGACATAGGTTCAACTGCATCAAAA |
| | ACAGTAGTATTAAAAAATGGTAAAGAAATTGTAAGTCAAGCAGT |
| | AATAAGTGTAGGGGCCGGAACAAGTGGCCCCAAGAGAGCTATAG |
| | ATTCTGTATTAAAAGATGCTAAATTATCCATTGAAGATTTAGAC |
| | TATATTGTATCCACTGGATATGGAAGAAATAGTTTCGATTTTGC |
| | TAACAAACAAATTTCTGAATTAAGTTGTCATGCAAAAGGGGTCT |
| | ATTTCGATAACAATAAAGCTAGAACAGTTATTGATATAGGCGGA |
| | CAAGATATTAAAGTATTAAAATTAGCGGATAGTGGAAGACTTTT |
| | AAACTTTATAATGAATGATAAATGTGCTGCAGGAACGGGACGAT |
| | TTTTAGATGTAATGTCTAGAGTAATAGAAGTTCCAGTTGATGAG |
| | TTAGGAAAAAAAGCATTAGAAAGCAAAAATCCTTGTACTATTAG |
| | TTCTACCTGTACAGTATTTGCAGAGTCAGAAGTAATTTCTCAAC |
| | TTGCAAGAGGAGTTAAAACTGAAGATTTGATAGCAGGAATTTGT |
| | AAATCTGTAGCATCAAGAGTGGCTAGCCTTGCAAAGAGAAGTGG |
| | TATAGAAGAATTAGTAGTTATGAGTGGAGGAGTAGCTAAAAATA |
| | TAGGTGTAGTAAAGGCAATGGAAGCAGAATTGGGAAGAGACATA |
| | TATATATCTAAAAATTCTCAATTAAATGGAGCATTGGGAGCAAG |
| | TCTATACGCTTATGAAAGTTTTCAAAAAGAAAGGAGCTAAAAAC |
| | ATGAGTGATAGAAATAAGGAAGTAAAAGAAAAAAAGGCAAAGCA |
| | TTATCTTAGAGAGATTACTGCAAAGCATTACAAAGAAGCTCTCG |
| | AAGCAAAAGAAAGGGGAGAAAAGGTTGGTTGGTGTGCATCTAAC |
| | TTCCCACAAGAAATAGCTACAACATTGGGGGTAAAAGTTGTTTA |
| | TCCAGAAAATCATGCAGCAGCTGTAGCAGCTAGAGGGAATGGAC |
| | AAAATATGTGTGAACATGCTGAGGCTATGGGTTTTTCTAATGAT |
| | GTATGTGGTTATGCAAGAGTAAATTTAGCTGTTATGGACATAGG |
| | TCATAGTGAAGATCAACCAATACCTATGCCAGACTTTGTACTTT |
| | GCTGTAATAACATTTGTAATCAAATGATTAAATGGTATGAGCAT |
| | ATAGCAAAAACTTTAGATATACCAATGATTCTTATAGATATACC |
| | ATACAATACAGAAAATACTGTTTCACAAGATAGAATTAAATATA |
| | TTAGAGCACAATTTGATGATGCAATAAAACAATTGGAAGAAATA |
| | ACAGGCAAAAAATGGGATGAAAATAAATTTGAAGAAGTTATGAA |
| | AATATCCCAAGAAAGTGCAAAACAATGGTTAAGAGCAGCATCCT |
| | ATGCAAAGTATAAACCTTCACCATTTAGCGGATTTGATTTATTT |
| | AATCATATGGCTGTAGCAGTTTGTGCAAGAGGTACACAAGAAGC |
| | TGCAGATGCATTTAAGATGTTAGCAGATGAATATGAGGAGAATG |
| | TAAAAACTGGAAAATCCACTTATAGGGGAGAAGAAAAACAACGT |
| | ATATTATTTGAAGGGATTGCCTGTTGGCCATATTTGAGACATAA |
| | ATTAACTAAGCTTAGTGAATATGGTATGAACGTAACTGCAACTG |
| | TATACGCAGAAGCCTTTGGTGTTATATATGAGAATATGGATGAA |
| | TTAATGGCTGCTTATAATAAAGTTCCTAATTCAATTAGTTTTGA |
| | AAACGCATTAAAAATGAGATTAAATGCTGTTACAAGCACTAATA |
| | CAGAAGGTGCTGTTATTCATATAAATAGAAGCTGTAAATTATGG |
| | AGTGGATTTTTATATGAGCTAGCAAGAAGATTAGAAAAGGAAAC |
| | AGGAATTCCTGTAGTATCATTTGATGGGGACCAGGCAGACCCAA |
| | GAAATTTCTCAGAAGCTCAATATGATACTAGAATTCAAGGACTT |
| | AATGAAGTAATGGTTGCTAAAAAGGAGGCTGAATAAGATGTCAA |
| | ATTCAGATAAATTTTTTAATGACTTTAAGGATATTGTAGAAAAT |
| | CCTAAAAAATATATAATGAAGCATATGGAACAAACTGGACAAAA |
| | GGCTATAGGATGTATGCCATTATATACTCCTGAGGAACTTGTAT |
| | TAGCTGCTGGAATGTTTCCAGTAGGGGTATGGGGAAGCAATACA |
| | GAACTTTCAAAAGCTAAAACATATTTCCCAGCATTTATTTGTTC |
| | AATATTACAAACAACATTGGAAAATGCATTAAATGGAGAATATG |
| | ATATGTTATCTGGTATGATGATTACAAATTATTGTGATTCATTA |
| | AAATGCATGGGACAAAATTTTAAACTAACCGTTGAAAATATTGA |
| | GTTTATCCCAGTAACAGTTCCACAAAATAGAAAAATGGAAGCTG |
| | GAAAAGAGTTTTTAAAAAGTCAATATAAAATGAATATTGAGCAA |
| | TTAGAAAAGATTTCTGGTAATAAAATAACAGATGAATCTTTAGA |
| | AAAAGCTATAGAAATATATGATGAACACAGAAAAGTAATGAATG |
| | ACTTTTCAATGTTAGCATCAAAATATCCAGGTATAATAACACCA |
| | ACTAAACGTAATTATGTTATGAAATCTGCTTATTATATGGATAA |
| | AAAAGAACATACTGAAAAAGTTAGACAATTAATGGATGAAATTA |
| | AAGCTATAGAACCAAAACCATTTGAAGGAAAGAGAGTTATAACT |
| | ACAGGTATAATTGCAGATTCAGAAGATTTACTTAAAATATTAGA |
| | AGAAAATAATATAGCTATAGTTGGTGATGATATAGCACATGAAT |
| | CTAGACAATATAGAACATTGACTCCAGAAGCGAACACACCAATG |
| | GATAGGTTAGCTGAGCAATTTGCTAATAGAGAATGTAGTACTTT |
| | ATATGATCCTGAAAAGAAAAGGGGTCAATATATAGTAGAAATGG |
| | CTAAAGAGAGAAAAGCAGATGGAATTATATTTTTCATGACAAAA |
| | TTCTGTGACCCAGAGGAATATGATTATCCACAAATGAAAAAGGA |
| | TTTTGAAGAAGCAGGCATTCCACATGTACTAATAGAAACTGATA |
| | TGCAAATGAAAAATTATGAACAAGCTAGAACTGCAATTCAGGCT |
| | TTTTCAGAAACACTTTAATAAAAGTTTTCAATATTTACTGTAAA |
| | CTTTATTAATTGAAACATTGATTTCTCTTCTCTTTCTATAAAAT |
| | AATATTTATATTTAAAAAAGTTATGTTTAGATGGATGAAAGGAA |
| | ATCAATGTTCATATAAATTAACAAATTCATTAATATATTAGGAG |
| | GGATATAATGTTTTTCACAGAACAACATGAACTTATTAGAAAAT |
| | TAGCAAGAGATTTTGCAGAGCAGGAAATAGAGCCTATTGCAGAT |
| | GAAGTAGATAAAACTGCCGAGTTCCCTAAAGAAATTGTGAAAAA |
| | AATGGCCCAAAATGGTTTTTTTGGAATAAAAATGCCTAAAGAAT |
| | ATGGTGGAGCTG |

Bacteria comprise nucleic acid sequences having a particular degree of homology or identity to other bacteria. The terms “identity,” “homology,” and “homologous” as used herein refer to a degree of complementarity or shared similarity with other nucleotide sequences. There may be partial homology or complete homology (i.e., identical sequences). A nucleotide sequence which is partially complementary, i.e., “substantially homologous” or “substantially identical” to a nucleic acid sequence is one that at least partially inhibits a completely complementary sequence from hybridizing to a target nucleic acid sequence.

In some aspects, bacteria of the disclosure comprise a nucleic acid sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homologous or identical to the nucleic acid sequence of SEQ ID NO: 1.

In some aspects, bacteria comprising the nucleic acid sequence of SEQ ID NO: 1 can be a probiotic or a probiotic bacterium. The term “probiotic” as used herein can mean one or more live microorganisms (e.g., bacteria or yeast) which, when administered appropriately, can confer a health benefit on the subject.

Some non-limiting examples of bacteria of the disclosure include, but are not limited to, Clostridium sporogenes, Peptostreptococcus anaerobius, Clostridium cadaveris, Clostridium boltae, and combinations thereof. Preferably, the bacteria are Clostridium sporogenes.

The composition can comprise a fermentate. The fermentate can be produced using any fermentation method known in the art. Particularly suitable fermentation methods are further described in U.S. Pat. Nos. 6,806,069, 6,864,231, 6,942,856, and 7,138,113, which are herein incorporated by reference in their entirety.

In some aspects, the fermentate can be produced by (1) fermenting a first microorganism in a fermentation medium, (2) adding a proteolytic enzyme(s) to break the cell walls of the first microorganism, (3) adding one or more second microorganisms and fermenting, (4) heating to deactivate the microorganisms, (5) homogenizing the mixture, and (6) spray drying to produce a powder fermentate.

In some aspects, the fermentate can be produced by (1) fermenting a first microorganism in a fermentation medium, (2) adding a proteolytic enzyme(s) to break the cell walls of the first microorganism, (3) adding one or more second microorganisms, (4) heating to deactivate the microorganisms, (5) homogenizing the mixture, and (6) spray drying to produce a powder fermentate.

In some aspects, a first microorganism can be added to a suitable medium that can allow microorganism growth and fermentation, preferably water, to form a fermentation medium. The number of CFUs of the first microorganism can vary based on the type of microorganism used. Preferably the first microorganism is a yeast such as Saccharomyces cerevisiae.

In some aspects, additional nutrients can be added to further induce the growth of the microorganisms and the fermentation. The additional nutrients can be added to the fermentation medium as individual ingredients or can be added to a nutrient medium, which is then added to the fermentation medium. Additional nutrients can include, for example, amino acids, carbohydrates, soy flour, nutritional yeast such as inactive baker's yeast or inactive brewer's yeast, and combinations thereof. Non-limiting examples of amino acids can include glutamine, lysine, cysteine, methionine, aspartic acid, leucine, valine, alanine, arginine, glycine, and combinations thereof. Carbohydrates can include polysaccharides, oligosaccharides, disaccharides, monosaccharides, and combinations thereof. Non-limiting examples of suitable carbohydrates can include maltose or gum acacia.

In some aspects, the fermentation medium can be maintained under conditions that promote optimal microorganism growth, such as between about 32.2° C. (90° F.) to about 35° C. (95° F.). The first microorganisms can be allowed to ferment for a sufficient amount of time, such as for about 4 hours.

In some aspects, one or more proteolytic enzymes can be added to the fermentation medium after the first microorganisms have fermented. Non-limiting examples of suitable proteolytic enzymes can include papain, bromelain, pepsin, or fungal protease. One advantage to including a proteolytic enzyme is that it can help to break down the cell wall of the first microorganisms. The amount of proteolytic enzyme can vary depending on the number of microorganisms in the fermentation medium. In some aspects, from about 1 to about 50 g of proteolytic enzyme can be added per 500 g of microorganism.

In some aspects, the one or more second microorganisms can optionally be added to the fermentation medium. Suitable second microorganisms can include lactic acid bacteria and Bifidobacteria, such as Lactobacillus acidophilus, Bifidobacterium bifidum, Lactobacillus rhamnosus, and combinations thereof. Preferably, the second microorganisms are added after the first microorganisms have fermented. In some aspects, the fermentation medium can be maintained at a suitable temperature and condition to allow the growth and fermentation of the second microorganisms. Such conditions are known in the art. Alternatively, the second microorganisms can be added after the first microorganisms have fermented but are not allowed to grow or ferment further before deactivation.

In some aspects, the microorganisms in the fermentation medium can be deactivated after fermentation. Preferably, the microorganisms can be deactivated by raising the temperature of the fermentation medium. For example, the microorganisms can be deactivated by heating the fermentation medium to about 65.6° C. (150° F.) to about 93.3° C. (200° F.), preferably about 71.1° C. (160° F.) to about 76.7° C. (170° F.), for approximately 30 minutes to about three hours with stirring.

In some aspects, the fermentation medium can be homogenized after fermentation in order to form a more uniform product. Methods of homogenization are known in the art, and can be performed, for example, by a homogenization pump, a shearing pump, or a blender.

In some aspects, the bacteria in the fermentation medium can be separated from the mixture, by centrifugation for example. Then the supernatant can be dehydrated to form a powder fermentate.

It is preferred that the fermentation medium be dehydrated after fermentation. Methods for dehydrating solutions are well known in the art and can include lypohilization, spray drying, open air drying, and drum drying. Preferable the fermentation medium is spray dried. After dehydrating the fermentation medium, a powder fermentate is formed which can then be incorporated into a dosage form or other form suitable for administration.

In some aspects, a stabilizing excipient or cryoprotectant can be added to the fermentation medium or supernatant prior to dehydration. In various aspects, the terms “stabilizing excipient” and “cryoprotectant” are used interchangeably herein. In some aspects, suitable cryoprotectants can include inositol, sorbitol, mannitol, trehalose, glucose, sucrose, corn syrup, DMSO, starches and/or modified starches of all types, Polyvinylpyrrolidone (PVP), maltose, or other mono and disaccharides.

In some aspects, the fermentate can comprise yeast. In some aspects, the fermentate can comprise yeast and one or more proteolytic enzymes. Alternatively, the fermentate can comprise yeast, one or more proteolytic enzyme, and optionally additional nutrients selected from the group consisting of carbohydrates, soy flour, and combinations thereof. Preferably, the fermentate can comprise gum arabic, soy flour, Saccharomyces cerevisiae, bromelain, papain, and combinations thereof. In some aspects, the bromelain and papain can be deactivated. In some aspects, the fermentate can contain organic ingredients. In some aspects, the S. cerevisiae can be inactivated.

In some aspects, the fermentate can further comprise lactic acid bacteria and/or Bifidobacteria, such as Lactobacillus acidophilus, Bifidobacterium bifidum, Lactobacillus rhamnosus, and mixtures thereof. In some aspects the lactic acid bacteria and/or Bifidobacteria can be inactivated.

In some aspects, the composition can comprise from about 1 mg to about 2 g of the fermentate, alternatively from about 10 mg to about 1.5 g, alternatively from about 25 mg to about 1 g. In some aspects, the composition can comprise from about 1 mg to about 500 mg of the fermentate, alternatively from about 15 mg to about 250 mg, alternatively from about 50 mg to about 150 mg.

In one aspect, the composition can comprise from about 0.01% to about 90% of the fermentate, alternatively from about 0.1% to about 85%, alternatively from about 1% to about 80%, alternatively from about 2.5% to about 75%, alternatively from about 5% to about 60%, alternatively from about 10% to about 50%, alternatively from about 15% to about 25%, all by weight of the composition. In some aspects, the composition can comprise bacteria from about 1×E3 to about 1×E13 CFU/g of fermentate.

In some aspects, the bacteria can produce at least about 1 μg/mL of IPA, alternatively at least about 2.5 μg/mL of IPA, alternatively at least about 5 μg/mL of IPA. Such amount or concentration of IPA is measured after anaerobic in vitro incubation of the bacteria at 36° C. with the fermentate described herein. For example, in some aspects, the IPA production disclosed herein above is measured over a period of about 12 hours, over a period of about 24 hours, over a period of about 36 hours, over a period of about 2 days, over a period of about 3 days, over a period of about 4 days, over a period of about 5 days, over a period of about 6 days, over a period of about a week, and the like. In particular aspects, the IPA is measured over a period of about 24 hours.

In some aspects, the bacteria can produce from about 5 to about 80 μg/mL of IPA after 24 hours of anaerobic in vitro incubation at 36° C. with the fermentate, alternatively from about 6 to about 50 μg/mL, alternatively from about 8 to about 25 μg/mL, alternatively from about 10 to about 15 μg/mL. In some aspects, the bacteria can produce from about 1 to about 80 μg/mL of IPA after 24 hours of anaerobic in vitro incubation at 36° C. with the fermentate, alternatively from about 1.5 to about 50 μg/mL, alternatively from about 4 to about 25 μg/mL, alternatively from about 6 to about 15 μg/mL.

In some aspects, the bacteria can produce other indole derivatives. As used herein, “other indole derivatives” refers to tryptophan derived indole metabolites including indole-3-acrylic acid, and indole-3-lactic acid, and indole-3-acetic acid.

In some aspects, the composition can comprise an excipient, carrier, and/or diluent. Nutritionally acceptable excipients, carriers or diluents include, but are not limited to, those suitable for human or animal consumption and those that are used standardly in the food industry. Typical nutritionally acceptable excipients, carriers or diluents are familiar to the skilled person in the art.

Examples of such suitable excipients for the various different compositions described herein, in some aspects, are found in the “Handbook of Pharmaceutical Excipients, 2nd Edition, (1994), Edited by A Wade and P J Weller. Acceptable carriers or diluents, in some aspects, are described, for example, in Remington's Pharmaceutical Sciences, Mack Publishing Co. (A. R. Gennaro edit. 1985). Such suitable carriers include, but are not limited to, methyl cellulose, magnesium stearate, and the like. Such suitable diluents include, but are not limited to water, ethanol, and glycerol.

The choice of pharmaceutical excipient, carrier, or diluent is selected with regard to the intended route of administration and standard pharmaceutical or nutraceutical practice. Such compositions, in some aspects, may comprise, in addition to the excipient, carrier or diluent, additional ingredients. Such additional ingredients include, but are not limited to, any suitable binder(s), lubricant(s), suspending agent(s), coating agent(s), solubilizing agent(s), preservatives, dyes, flavoring agent(s), and/or suspending agents.

Examples of suitable binders include, but are not limited to, starch, gelatin, natural sugars, and combinations thereof. Such natural sugars include, but are not limited to, glucose, anhydrous lactose, free-flow lactose, beta-lactose, corn sweeteners, and natural and/or synthetic gums, such as acacia, tragacanth or sodium alginate, carboxymethyl cellulose and polyethylene glycol. Examples of suitable lubricants include, but are not limited to, sodium oleate, sodium stearate, magnesium stearate, sodium benzoate, sodium acetate, sodium chloride, and combinations thereof. Preservatives, stabilizers, dyes, and flavoring agents, in some aspects, are also provided in the composition. Examples of preservatives include, but are not limited to, sodium benzoate, sorbic acid, esters of p-hydroxybenzoic acid, and combinations thereof. In some aspects, suspending agents may also be present in the composition.

In some aspects, the composition can optionally comprise one or more active ingredients. The active ingredients can include vitamins, minerals, prebiotics, glycans (e.g., as decoys that would limit specific bacterial/viral binding to the intestinal wall), and combinations thereof. Non-limiting examples of active ingredients can include vitamin C, vitamin D, vitamin E, vitamin K1, Vitamin K3, vitamin B1, vitamin B3, folic acid, vitamin B12, vitamin B3, vitamin B7, pantothenic acid, calcium, magnesium, iron, iodide, zinc, copper, manganese, chromium, molybdenum, beta-carotene, melatonin, and combinations thereof.

The term “prebiotic” as used herein can be a general term to refer to chemicals and/or ingredients that can affect the growth and/or activity of microorganisms in a subject or host (e.g., can allow for specific changes in the composition and/or activity in the microbiome) and can confer a health benefit on the subject. Prebiotics include, but are not limited to, complex carbohydrates, complex sugars, resistant dextrins, resistant starch, amino acids, peptides, nutritional compounds, biotin, polydextrose, fructooligosaccharide (FOS), galactooligosaccharides (GOS), inulin, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, and xylooligosaccharides (XOS). In some aspects, anti-oxidant ingredients, such as, e.g., vitamin C, are included as prebiotic substrates to act as oxygen scavengers. Prebiotic substrates, such as these, improve the colonization and survival of the bacteria in vivo. Prebiotics, in some aspects, are selectively fermented, e.g., in the colon.

Prebiotics, in various aspects, are found in foods (e.g., acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. In some aspects, prebiotics are administered in other forms (e.g. capsule or dietary supplement).

The active ingredients can be at levels above, below, and/or equal to the recommended daily allowance (“RDA”), depending on the particular active ingredient. Exemplary RDA values for numerous nutritional compounds are listed in 21 CFR 101 and further RDA values are also published by the Institute of Medicine of the National Academy of Science. In some aspects, the active ingredient is present in an amount from about 0.01 to about 50% by weight, with respect to the total weight of the composition. In some aspects, the active ingredient can be present in an amount from about 0.1 to about 40% by weight, alternatively from about 1 to about 30%, alternatively from about 3 to about 25%, alternatively from about 5 to about 20%. In some aspects, the active ingredient can be present in an amount of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50%.

The composition can optionally comprise one or more herbal ingredients. Non-limiting examples of herbal ingredients can include rosemary (leaf), ginger, lemon balm, green tea, holy basil, oregano, thyme, ashwagandha, bacopa, chamomile, valerian, and combinations thereof. In some aspects, the composition comprises ashwagandha. In some aspects, the herbal ingredient can be whole herbs or plant parts, extracts, powders, concentrates, or combinations thereof. In some aspects, the herbal ingredient can be supercritical extracts and/or hydroalcoholic extracts. As used herein, the term “supercritical extraction” refers to the technique in which hydrophobic compounds can be extracted from samples utilizing a supercritical fluid. The solvation power of a supercritical fluid is increased as the pressure and temperature are increased above their critical points, producing an effective solvent for the isolation of hydrophobic molecules. In some aspects, the herbal ingredients can be fermented using methods known to one of skill in the art. The fermented herbal ingredients can be prepared by collecting the supernatants of the herbal fermentations and drying the mixture by any known method in the art, such as spray-drying. The culture media can contain ingredients selected from the group consisting of organic milled soy, Saccharomyces cerevisiae (organic yeast: active and inactive), organic maltodextrin, organic gum acacia, organic orange peel, organic lemon peel, organic carrot powder, organic alfalfa powder, Lactobacilli (L. acidophilus, L. bifidus, L. rhamnosus) and enzymes (deactivated), and combinations thereof. The fermented herbal ingredients can contain all or some of the ingredients from the culture media.

In some aspects, the composition can comprise from about 0.1 to about 10% of the one or more herbal ingredients, alternatively from about 1 to about 8%, alternatively from about 2 to about 6%, all by weight of the composition.

In some aspects, the composition can be substantially free of vitamins, minerals, and/or herbs which inhibit IPA production. In some aspects, the composition can be substantially free of Vitamin B2, selenium, and/or Vitamin B6. As used herein, “substantially free of” means containing less than about 0.1%, by weight of the composition, alternatively less than about 0.05% alternatively less than about 0.01%, alternatively less than about 0.001%.

The composition can be in any dosage form known in the art. Some non-limiting examples of dosage forms can include topical, capsule, pill or tablet, gummy, soft chew, panned chew, sachet, gel, liquid, bulk powder for reconstitution or a drink prepared from bulk powder, and the like. In some aspects, the composition can be incorporated into a form of food and/or drink. Non-limiting examples of food and drinks where the composition is incorporated can include bars, shakes, juices, beverages, frozen food products, fermented food products, and cultured dairy products such as yogurt, yogurt drink, cheese, acidophilus drinks, and kefir.

In some aspects, the composition may be in the form of a dietary supplement or a pharmaceutical composition. As used herein, the term “dietary supplement” refers to a composition intended to supplement a diet of food and water, where the diet is sufficient to support life.

In some aspects, the composition can comprise an amount of the one or more bacteria and fermentate effective to provide a health benefit to a subject. In some aspects, the effective amount is a therapeutically effective amount.

In some aspects, a composition can be formulated such that the one or more of the bacteria present in the composition can replicate once they are delivered to the target habitat (e.g., the gut). In one non-limiting example, the composition is formulated in a pill, powder, capsule, tablet, enteric-coated dosage form or package, such that the composition has a shelf life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In some aspects, other components are added to the composition to aid in the shelf life of the composition. In some aspects, one or more bacteria may be formulated in a manner allowing survival in a non-natural environment. For example, bacteria that is native to the gut may not survive in an oxygen-rich environment. To overcome this limitation, the bacteria may be formulated in a pill or package that can reduce or eliminate the exposure to oxygen. Other strategies to enhance the shelf-life of bacteria may include other microbes (e.g., if the bacterial consortia comprise a composition whereby one or more strains are helpful for the survival of one or more strains).

In some aspects, the composition can be formulated as a powder, tablet, capsule, enteric-coated dosage form (e.g., for delivery to ileum/colon), or pill that can be administered to a subject by any suitable route. The lyophilized formulation can be mixed with a saline or other solution prior to administration.

In some aspects, the composition is formulated for oral administration. In some aspects, the composition is formulated as a powder, tablet, capsule, enteric-coated dosage form or pill for oral administration. In some aspects, the composition is formulated for delivery of the bacteria to the ileum region of a subject. In some aspects, the composition is formulated for delivery of the bacteria to the colon region (e.g., upper colon) of a subject. In some aspects, the composition is formulated for delivery of the bacteria to the ileum and colon regions of a subject.

An enteric coating can protect the contents of the oral formulation, for example, tablet or capsule, from the acidity of the stomach and provide delivery to the ileum and/or upper colon regions. Non-limiting examples of enteric coatings can include pH sensitive polymers (e.g., Eudragit® FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, plant fibers, and combinations thereof. In some aspects, the enteric coating is formed by a pH sensitive polymer. In some aspects, the enteric coating is formed by Eudragit® FS30D.

In some aspects, the enteric coating can be designed to dissolve at any suitable pH. In some aspects, the enteric coating can be designed to dissolve at a pH greater than about pH 5.0, or at a pH greater than about pH 6.0, or at a pH greater than about pH 7.0. In some aspects, the enteric coating can be designed to dissolve at a pH greater than about pH 5.0 to about pH 7.0. In some aspects, the enteric coating can be designed to dissolve at a pH greater than about pH 5.0, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5.

Formulations provided herein can include the addition of one or more agents to the composition in order to enhance stability and/or survival of the microbial formulation. Non-limiting example of stabilizing agents can include genetic elements, glycerin, ascorbic acid, skim milk, lactose, tween, alginate, xanthan gum, carrageenan gum, mannitol, palm oil, poly-L-lysine (POPL), and combinations thereof.

In some aspects, the composition can be formulated into unit dosage form, i.e., in the form of discrete portions containing a unit dose, or a multiple dose, or a sub-unit of a unit dose. For example, a typical or usual suitable or effective dose in humans of the one or more bacteria is from about 1×E3 (1×E3=1×10\hat{ }3=1×(10 to the power 3)) to about 1×E13 colony forming units (CFU). In some instances, a suitable or effective dose can be from about 1×E6 to about 1×E11 CFU. In particular instances, a suitable or effective dose can be from about 1×E7 to about 1×E10 CFU. In some additional aspects, a suitable or effective dose of the bacteria can be about 1×E2 CFU, 1×E3 CFU, 1×E4 CFU, 1×E5 CFU, 1×E6 CFU, 1×E7 CFU, 1×E8 CFU, 1×E9 CFU, 1×E10 CFU, 1×E11 CFU, 1×E12 CFU, 1×E13 CFU, 1×E14 CFU, or 1×E15 CFU.

The composition can be administered once daily. Alternatively, the composition can be taken twice daily, alternatively three times daily, alternatively four times daily. The composition can be taken with meals or on an empty stomach. The composition can be taken in the morning, mid-day, afternoon, evening, or at night. The composition can be taken at the same time every day or the time the composition is taken can vary. A user can administer one dosage form per dose of the composition, in another example two dosage forms, in another example three dosage forms, in another example four dosage forms, and in another example more than four dosage forms. In some aspects, the dose is about 0.1 milligrams (mg), about 0.2 mg, about 0.3 mg., about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5.0 mg, about 6.0 mg, about 7.0 mg, about 8.0 mg, about 9.0 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 300 mg, about 400 mg, about 500 mg, about 600 mg, about 700 mg, about 800 mg, about 900 mg, or about 1 gram. In some aspects, a dose ranges from about 1 mg to about 500 mg.

In some aspects, the composition can comprise a prebiotic, and a dose of the composition can be from about 50 mg to about 5 g, alternatively from about 100 mg to about 4 g, alternatively from about 250 mg to about 2 g.

In some aspects, the composition can comprise one or more bacteria in an amount of from about 1×E3 to about 1×E13 colony forming units (CFU)/gram (g), with respect to the weight of the composition. In some aspects, one or more bacteria can be present in an amount from about 1×E5 to about 1×E11 CFU/g. In some aspects, one or more bacteria can be present in an amount from about 1×E6 to about 1×E10 CFU/g. In some aspects, one or more bacteria can be present in the composition in an amount from about 1×E8 to about 1×E10 CFU/g. In some aspects, a composition can comprise one or more bacteria present in an amount of about 1×E1 CFU/g, about 1×E2 CFU/g, about 1×E3 CFU/g, about 1×E4 CFU/g, about 1×E5 CFU/g, about 1×E6 CFU/g, about 1×E7 CFU/g, about 1×E8 CFU/g, about 1×E9 CFU/g, about 1×E10 CFU/g, about 1×E11 CFU/g, about 1×E12 CFU/g, about 1×E13 CFU/g, about 1×E14 CFU/g, or about 1×E15 CFU/g.

Suitable containers for use with the composition described herein can include, for example, cans, jars, bottles, bottles with shaker lids, mills, vials, syringes, tubes, pouches, sachets, bags, blister cards, or folders. The containers can be formed from a variety of materials including without limitation glass, plastic, polymers, metals, alloys, metal or alloy foil, rubber, cardboard, or paper. The containers can also comprise a sealant, which can be formed from any material suitable in the art such as a resin or polymer. The container can comprise a moisture barrier and/or oxygen barrier to further enhance the viability of the probiotics during storage. Moisture barriers and oxygen barriers are known in the pharmaceutical and food industries. Suitable barriers for use in the present invention are described in U.S. Pat. No. 6,716,499 to Vadhar, U.S. Pat. No. 6,524,720 to Shah, U.S. Pat. No. 5,792,530 to Bonner et al., and U.S. Pat. No. 4,977,004 to Bettie et al. In addition to, or in lieu of such barriers, the containers may comprise an oxygen scavenger and/or a desiccant/moisture absorbing compound.

Suitable oxygen scavengers and desiccants are known in the art, for example, U.S. Pat. No. 6,746,622 to Yan et al., U.S. Pat. No. 6,387,461 to Ebner et al., and U.S. Pat. No. 6,228,284 to Ebner et al., and U.S. Pat. No. 6,130,263 to Hekal.

Also described herein are methods of providing one or more health benefits comprising orally administering the present composition to a user. In some aspects, the one or more health benefits may be selected from the group consisting of promoting brain health; promoting healthy aging of the brain;

promoting emotional well-being via brain health; delivering antioxidant nutrients to the brain;

managing oxidative stress in the brain; reducing and/or maintaining oxidative stress or total antioxidant capacity in the brain; protecting neurons via delivering antioxidants; and any combination of the foregoing. In some aspects, the one or more health benefits may be selected from the group consisting of promoting brain health; promoting healthy aging of the brain; delivering antioxidant nutrients to the brain; managing oxidative stress in the brain; and any combination of the foregoing.

Also described herein are methods of increasing IPA in the gastrointestinal tract and/or serum of a subject in need thereof comprising administering to the subject an effective amount of the composition described herein.

Also described herein are methods for optimizing the gut-brain axis for a healthy nervous system via reducing neuroinflammation and neurodegeneration of a subject in need thereof comprising administering to the subject an effective amount of the composition described herein.

Also described herein are methods for treating, ameliorating, or preventing a disorder in a subject suffering therefrom or at risk of suffering therefrom comprising administering to the subject an effective amount of the composition described herein. In some aspects, the disorder can be an intestinal disorder, a metabolic disorder, an inflammatory disorder, or an immune disorder. In some aspects, the disorder can be a metabolic syndrome, insulin resistance, insulin sensitivity, pre-diabetes, diabetes, anxiety, depression, autism, hypertension, irritable bowel syndrome, metabolism irregularity, stress-related conditions, neurological disorders, such as Parkinson's disease, Inflammatory Bowel Disease (IBD), Crohn's Disease, heart disease, or a nervous system disorder such as multiple sclerosis.

### Fermentate Test

Different fermentate compositions were incubated with C. sporogenes to assess the effect on IPA production. In Samples 1-11, different fermentate powders prepared by fermenting fermentation media (described in Table 1) or unfermented control media were incubated with C. sporogenes and IPA production was measured. Fermentate powders were obtained from Pharmachem Laboratories, Kearny, N.J. Sample 10 was a positive control in which C. sporogenes was incubated with Peptone Yeast Glucose media (commercially available from Sigma-Aldrich, St. Louis, Mo.). Sample 11 was a tryptophan control, in which C. sporogenes was incubated with a solution containing tryptophan, vitamins, and trace elements. Sample 11 was used as a control to assess the impact of tryptophan (the substrate for the bacteria to make IPA) in the fermentate on IPA production. Sample 11 contained 150 μg/mL tryptophan, which corresponds to the level of tryptophan measured in the fermentate powders in Samples 1-9. It is believed that during fermentation of the media, tryptophan is produced and can be measured in the final fermentate composition.

C. sporogenes ATCC 15579 was grown anaerobically at 36° C. for 24 hours in 10 mL of Peptone Yeast Glucose (“PYG”) media (commercially available from Sigma-Aldrich, St. Louis, Mo.). A 10 mL sample of the 24 hr culture (approximately 1×E8/mL) was centrifuged at 10,0000×g for 5 min. The supernatant was removed and the C. sporogenes pellet was resuspended in 10 mL of saline to wash the bacteria. The sample was then centrifuged at 10,000×g for 5 min. The supernatant was removed and the C. sporogenes pellet was resuspended in 10 mL of saline to create an inoculum preparation. 10 mL of saline was added to each of 22 sterile glass tubes. In duplicate, 1% (0.1 grams) of fermentate was added to one of the glass tubes.

100 μl of the inoculum preparation was then transferred into each of the glass tubes anaerobically. 100 μl of C. sporogenes was transferred into 10 mL of PYG media anaerobically as a positive control (Sample 10). 100 μl of C. sporogenes was transferred into 10 mL of basal media containing 150 μg/mL tryptophan, 1% Vitamin Supplement ATCC® MD-VS™ (commercially available from ATCC, Manassas, Va.) and 1% Trace Mineral Supplement ATCC® MD-TMS™ (commercially available from ATCC, Manassas, Va.) anaerobically as a tryptophan control (Sample 11). Then, the glass tubes were transferred into a 36° C. box in the anaerobic chamber for 24-28 hours. After incubation, all tubes were removed from the chamber and centrifuged at 8,000×g. The supernatant was removed and filtered through a 0.2 μm syringe filter into a sterile glass tube. 0.5 mL of each supernatant was placed in a 2.2 mL deep well plate in duplicate. The plate was then sealed and wrapped in foil until IPA analysis was performed. IPA was measured according to the IPA Measurement Method described hereafter.

Table 1 summarizes the results from this test. Samples 1-10 and 11 were tested at different times following the same protocol; however, the data are shown together for ease of comparison.

TABLE 1

| | | Average | |
|---|---|---|---|
| | | IPA | |
| Sample | Media Components | (μg/mL) | STDEV |
| 1 | Gum arabic, soy flour, Saccharomyces cerevisiae [active and | 10.60 | 0.28 |
| | inactive], bromelain, papain, Lactobacillus | | |
| | acidophilus, Bifidobacterium bifidum, and Lactobacillus | | |
| | rhamnosus | | |
| 2 | Gum arabic, soy flour, Saccharomyces cerevisiae [active and | 9.76 | 0.28 |
| | inactive], bromelain, and papain | | |
| 3 | Vitamin B2, gum arabic, soy flour, Saccharomyces cerevisiae | 5.11 | 0.21 |
| | [active and inactive], bromelain, papain, Lactobacillus | | |
| | acidophilus, Bifidobacterium bifidum, and Lactobacillus | | |
| | rhamnosus | | |
| 4 | Vitamin B2, gum arabic, soy flour, Saccharomyces cerevisiae | 4.97 | 0.04 |
| | [active and inactive], bromelain, and papain | | |
| 5 | Selenium, gum arabic, soy flour, Saccharomyces cerevisiae | 1.96 | 0.08 |
| | [active and inactive], bromelain, papain, Lactobacillus | | |
| | acidophilus, Bifidobacterium Bifidum, and Lactobacillus | | |
| | rhamnosus | | |
| 6 | Selenium, gum arabic, soy flour, Saccharomyces cerevisiae | 1.73 | 0.01 |
| | [active and inactive], bromelain, and papain | | |
| 7 | Vitamin A, vitamin C, vitamin D3, vitamin E, vitamin K, thiamin, | 0.66 | 0.02 |
| | riboflavin, niacin, vitamin B6, folate, vitamin B12, biotin, | | |
| | pantothenic acid, calcium, iron, iodine, magnesium, zinc, | | |
| | selenium, copper, manganese, chromium, molybdenum, Stress | | |
| | and Energy Support Blend (Organic Schizandra, Organic Maca, | | |
| | Organic Chamomile), Hormone Support Blend (Organic chaste | | |
| | tree, organic red clover, organic raspberry), Immune Support | | |
| | Blend (European Elder extract, Organic eleuthero, Organic | | |
| | astragalus), ginger hydroethanolic extract, organic turmeric | | |
| | powder, organic ginger supercritical extract, organic turmeric | | |
| | supercritical extract, gum arabic, soy flour, Saccharomyces | | |
| | cerevisiae [active and inactive], bromelain, papain, Lactobacillus | | |
| | acidophilus, Bifidobacterium bifidum, and Lactobacillus | | |
| | rhamnosus¹ | | |
| 8 | Vitamin B6, gum arabic, soy flour, Saccharomyces cerevisiae | 0.663 | 0.11 |
| | [active and inactive], bromelain, and papain | | |
| 9 | Vitamin B6, gum arabic, soy flour, Saccharomyces cerevisiae | 0.53 | 0.09 |
| | [active and inactive], bromelain, papain, Lactobacillus | | |
| | acidophilus, Bifidobacterium bifidum, and Lactobacillus | | |
| | rhamnosus | | |
| 10 (Positive | Peptone Yeast Glucose (“PYG”) media | 4.74 | 0.67 |
| control) | | | |
| 11 (Tryptophan | Basal media containing 150 μg/mL tryptophan, 1% Vitamin | 0.28 | 0.08 |
| Control) | Supplement ATCC ® MD-VS ™ and 1% Trace Mineral | | |
| | Supplement ATCC ® MD-TMS ™ | | |

¹Fermentate powder prepared in the same way as found in commercially available Everyday Women's One Daily Multivitamin product (New Chapter, Inc., Brattleboro, VT).

It was found that C. sporogenes produced elevated levels of IPA after incubation with specific fermentate compositions. Samples 1 and 2 had IPA levels of 10.60 μg/mL and 9.76 μg/mL, respectively, at least 30-fold higher than Sample 11 (Tryptophan Control). Without being limited by theory, it is believed that the fermentate compositions in Samples 1 and 2 provide growth factors that significantly boost IPA production.

A positive control was also included in the experimental design to confirm findings from previous literature that C. sporogenes indeed makes IPA in a growth medium such as PYG. In this case the IPA production was 4.74 μg/mL (Example 10), less than half the IPA produced in Samples 1 and 2.

Fecal Material Test

The fermentate composition tested in Sample 1 above was incubated with C. sporogenes in fecal material to assess the effect on IPA production. Since it was observed that the fermentate composition in Sample 1 yielded the highest amounts of IPA with a pure culture of C. sporogenes, the objective was to determine if incubation of C. sporogenes with the same fermentate would still yield elevated levels of IPA in the background of complex fecal microbial communities.

C. sporogenes ATCC 15579 was grown anaerobically at 36° C. for 24 hours in 10 mL of PYG media. A 10 mL sample of the 24 hr culture (approximately 1×E8/mL) was centrifuged at 10,0000×g for 5 min. The supernatant was removed and the C. sporogenes pellet was resuspended in 10 mL of saline to wash the bacteria. The sample was then centrifuged at 10,000×g for 5 min. The supernatant was removed and the C. sporogenes pellet was resuspended in 10 mL of saline to create an inoculum preparation.

Fecal samples from 28 individual donors were used in this assay. Samples had been previously aliquoted at approximately 1 gram each and frozen at −80° C. For this assay, 4 tubes of fecal aliquots from each donor were thawed and added to 40 mL of saline media. The samples were vortexed vigorously for 2 minutes. From this, 10 mL of each fecal solution was aliquoted into 4 separate sterile glass test tubes and were labeled “Fecal only” (Sample 12), “Fecal+C. sporogenes” (Sample 13), “Fecal+fermentate” (Sample 14), and Fecal+C. sporogenes+fermentate” (Sample 15). “Fecal+C. sporogenes” tubes had 100 μl of C. sporogenes added as prepared above. “Fecal+fermentate” tubes had 0.1 grams of fermentate added. “Fecal+C. sporogenes+fermentate” had 0.1 grams of fermentate and 100 μl of C. sporogenes added as prepared above.

After preparation, the samples were transferred into a 36° C. box in the anaerobic chamber for 24-28 hours. After incubation, all samples were removed from the chamber and centrifuged at 8,000×g. The supernatant was removed and filtered through a 0.2 μm syringe filter into a sterile glass tube. 0.5 mL of each supernatant was placed in a 2.2 mL deep well plate in duplicate. The plate was then sealed and wrapped in foil until IPA analysis was performed. IPA was measured according to the IPA Measurement Method described hereafter.

Table 3 summarizes the results from this test.

TABLE 3
IPA production in fecal material

| | | Fecal | | Average |
|---|---|---|---|---|
| Sample | C. sporogenes | Material | Fermentate | IPA (μg/mL) |
| 12 | No | Yes | None | 0.18² |
| 13 | No | Yes | A | 0.25³ |
| 14 | Yes | Yes | None | 2.51 |
| 15 | Yes | Yes | A | 4.76 |

²Average of the 4 replicates that had detectable IPA.
³Average of the 6 replicates that had detectable IPA.

It was found the addition of C. sporogenes in combination with the fermentate from Sample 1 above in fecal material can produce elevated levels of IPA. Sample 14 (C. sporogenes+Fecal material) had an average IPA level of 2.51 μg/mL. Sample 15 (C. sporogenes+Fecal material+fermentate) had an average IPA level of 4.76 μg/mL. It is expected that the fermentate from sample 2 above would perform similarly when added to a fecal sample with C. sporogenes present.

### IPA Measurement Method

Biological samples were subjected to protein precipitate by adding 300 μL of MeOH to 100 μL of sample. Samples were vortexed and centrifuged for 10 minutes at 3000 rpm using a benchtop centrifuge such as a Beckman Coulter Allegra® X15R (Rotor SX4750A), or equivalent, to pellet the protein and other precipitates. 150 μL of supernatant was transferred to a 96-well deep well plate along with 30 μL of 10 ng/mL Indole-3-Propionic Acid-2,2-d2 (IPA-d2) and 150 μL of water. For samples in other matrices including, but not limited to, bacterial cell culture filtrates and fermentates, samples were subjected to 1000-fold dilution with 10% MeOH in water. 30 μL of 10 ng/mL IPA-d2 were added to 300 μL of the diluted sample. The IPA and IPA-d2 in the isolated/diluted samples were subjected to gradient High-Performance Liquid Chromatography (HPLC) analysis on a Waters Atlantis T3 column, from Waters Corp., Milford, Mass., or equivalent, (2.1×50 mm, 3 μm particles), 0.1% formic acid in Water as mobile phase A and 0.1% formic acid in acetonitrile as mobile phase B. Detection and quantitation were achieved by tandem mass spectrometry operating under multiple reaction monitoring (MRM) MS/MS conditions (m/z 190.1130.0 for IPA, m/z 192.1→130.0 for IPA-d2). IPA calibration standards (STD), prepared in 10% MeOH in water, were used to construct a regression curve by plotting the response (peak area IPA/peak area IPA-d2) versus concentration for each standard. The concentrations of IPA in samples were determined by interpolation from the quadratic (1/x²) regression curve.

### Examples

The following examples further describe and demonstrate embodiments within the scope of the invention. The examples are given solely for the purpose of illustration and are not to be construed as limitations of the present invention, as many variations thereof are possible without departing from the spirit and scope of the invention.

The following compositions can be prepared in accordance with the present invention:

| | Example 1 | Example 2 | Example 3 | Example 4 | Example 5 |
|---|---|---|---|---|---|
| Ingredient | Wt % | Wt % | Wt % | Wt % | Wt % |
| C. Sporogenes | 5.0 | 5.0 | 5.0 | 4.0 | 6.0 |
| (1E11 CFU/g) | | | | | |
| Fermentate A | 0 | 0 | 10.0 | 10.0 | 10.0 |
| Fermentate B | 15.0 | 10.0 | 0 | 0 | 0 |
| Microcrystalline | 10.0 | 50.0 | 83.5 | 84.5 | 82.5 |
| Cellulose | | | | | |
| Hydroxypropylmethyl | 69.5 | 34.0 | 0 | 0 | 0 |
| Cellulose | | | | | |
| Magnesium Stearate | 0.5 | 1.0 | 1.5 | 1.5 | 1.5 |

| | Example 6 | Example 7 | Example 8 | Example 9 |
|---|---|---|---|---|
| Ingredient | Wt % | Wt % | Wt % | Wt % |
| C. Sporogenes | 5.0 | 5.0 | 5.0 | 5.0 |
| (1E11 CFU/g) | | | | |
| Fermentate A | 15.0 | 0 | 10.0 | 0 |
| Fermentate B | 0 | 10.0 | 0 | 10.0 |
| Microcrystalline | 70.0 | 42.0 | 74.0 | 10.0 |
| Cellulose | | | | |
| Hydroxypropylmethyl | 9.0 | 42.0 | 10.0 | 74.0 |
| Cellulose | | | | |
| Magnesium Stearate | 1.0 | 1.0 | 1.0 | 1.0 |

Examples 1-9 can be made according to the following method.

Fermentate A can be prepared by fermenting a fermentation medium containing gum arabic, soy flour, Saccharomyces cerevisiae [active and inactive], bromelain, papain, Lactobacillus acidophilus, Bifidobacterium bifidum, and Lactobacillus rhamnosus according to the fermentation method described in U.S. Pat. No. 6,806,069.

Fermentate B can be prepared by fermenting a fermentation medium containing gum arabic, soy flour, Saccharomyces cerevisiae [active and inactive], bromelain, and papain according to the fermentation method described in U.S. Pat. No. 6,806,069.

The resulting fermentation product can be dehydrated by spray drying to form a powdered fermentate. Alternatively, the fermentation product can be sprayed into liquid nitrogen to produce frozen beads. The frozen beads can be dried by lyophilization followed by milling to produce powdered fermentate.

The powdered fermentate can be weighed and loaded into a powder blender, such as a suitably sized “V” blender. C. sporogenes can then be weighed and loaded into the powder blender. Microcrystalline cellulose (USP) and hydroxypropylmethyl cellulose (USP, Hypromellose) (if present in the formulation) can be separately sieved, weighed, and loaded into the powder blender. Blending can be carried out until a homogeneous blend of fermentate, C. sporogenes, and excipients is obtained, typically mixing can be carried out for 100-500 revolutions. Magnesium stearate (USP) can be sieved and loaded into the powder blender. The magnesium stearate can be incorporated into the fermentate powder by blending for typically less than 100 rotations.

The final blend can be loaded into the powder feed hopper of a rotary encapsulator equipped with a capsule polisher. Gelatin or hydroxypropylmethyl cellulose capsules can be loaded into the capsule hopper. Capsules can be filled with the final blend and polished. Alternatively, the final blend can be loaded into a sachet filler equipped with a sachet sealer and the sachet material can be loaded. Sachets can be filled and sealed.

### Combinations

A. A composition comprising: (a) one or more bacteria having a nucleic acid sequence with at least 80% homology to the nucleic acid sequence of SEQ ID NO: 1; (b) a fermentate comprising a yeast; and (c) an excipient, carrier, and/or diluent.
B. The composition of paragraph A, wherein the one or more bacteria are selected from the group consisting of Clostridium sporogenes, Peptostreptococcus anaerobius, Clostridium cadaveris, Clostridium boltae, and combinations thereof.
C. The composition of paragraph A or B, wherein the fermentate further comprises one or more proteolytic enzymes.
D. The composition of any of paragraphs A-C, wherein the fermentate further comprises additional nutrients selected from the group consisting of carbohydrates, soy flour, and combinations thereof.
E. The composition of any of paragraphs A-D, wherein the fermentate further comprises at least one lactic acid bacteria or Bifidobacteria.
F. The composition of any of paragraph E, wherein the lactic acid bacteria or Bifidobacteria is selected from the group consisting of Lactobacillus acidophilus, Bifidobacterium bifidum, Lactobacillus rhamnosus, and mixtures thereof.
G. The composition of any of paragraphs A-F, wherein the composition comprises 1×E3 to 1×E11 colony-forming units (CFU) of the one or more bacteria.
H. The composition of any of paragraphs A-G, wherein the composition comprises from 1 mg to 2 g of the fermentate.
I. The composition of any of paragraphs A-H, wherein the one or more bacteria produce at least 5 μg/mL of indole-3-propionic acid (IPA) after 24 hours of anaerobic in vitro incubation at 36° C. with the fermentate.
J. The composition of any of paragraphs A-I wherein the composition further comprises an active ingredient.
K. The composition of any of paragraphs A-J wherein the composition further comprises an herbal ingredient.
L. The composition of any of paragraphs A-K, wherein the composition is a probiotic composition.
M. The composition of any of paragraphs A-L, wherein the one or more bacteria is Clostridium sporogenes.
N. A method of promoting brain health comprising administering to an individual in need thereof the composition of paragraph A.
O. A method of delivering antioxidant nutrients to the brain comprising administering to an individual in need thereof the composition of paragraph A.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Values disclosed herein as ends of ranges are not to be understood as being strictly limited to the exact numerical values recited. Instead, unless otherwise specified, each numerical range is intended to mean both the recited values and any real numbers including integers within the range. For example, a range disclosed as “1 to 10” is intended to mean “1, 2, 3, 4, 5, 6, 7, 8, 9, and 10” and a range disclosed as “1 to 2” is intended to mean “1.1, 1.2, 1.3, 1.4, 1.5, 1.6, 1.7, 1.8, 1.9, and 2.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
a. one or more bacteria having a nucleic acid sequence with at least 80% homology to the nucleic acid sequence of SEQ ID NO: 1;
b. a fermentate comprising soy flour, a yeast, and a proteolytic enzyme; and
c. an excipient, carrier, and/or diluent.

2. The composition of claim 1, wherein the one or more bacteria is selected from the group consisting of Clostridium sporogenes, Peptostreptococcus anaerobius, Clostridium cadaveris, Clostridium boltae, and combinations thereof.

3. The composition of claim 1, wherein the one or more bacteria is Clostridium sporogenes.

4. The composition of claim 1, wherein the fermentate further comprises a carbohydrate.

5. The composition of claim 1, wherein the fermentate further comprises at least one lactic acid bacteria or Bifidobacteria.

6. The composition of claim 5, wherein the lactic acid bacteria or Bifidobacteria is selected from Lactobacillus acidophilus, Bifidobacterium bifidum, Lactobacillus rhamnosus, and mixtures thereof.

7. The composition of claim 1, wherein the composition comprises from about 1×E3 to about 1×E11 colony-forming units (CFU) of the one or more bacteria.

8. The composition of claim 7, wherein the composition comprises from about 1 mg to about 2 g of the fermentate.

9. The composition of claim 1, wherein the one or more bacteria produce at least about 5 μg/mL of indole-3-propionic acid (IPA) after 24 hours of anaerobic in vitro incubation at 36° C. with the fermentate.

10. The composition of claim 1 wherein the composition further comprises an active ingredient.

11. The composition of claim 1 wherein the composition further comprises an herbal ingredient.

12. The composition of claim 1 wherein the composition is a probiotic composition.

13. A method of promoting brain health comprising administering to an individual in need thereof the composition of claim 1.

14. A method of delivering antioxidant nutrients to the brain comprising administering to an individual in need thereof the composition of claim 1.

15. A composition comprising:
a. one or more bacteria having a nucleic acid sequence with at least 80% homology to the nucleic acid sequence of SEQ ID NO: 1;
b. a fermentate comprising a yeast; and
c. an excipient, carrier, and/or diluent;
wherein the one or more bacteria produce from about 5 to about 80 μg/mL of indole-3-propionic acid (IPA) after 24 hours of anaerobic in vitro incubation at 36° C. with the fermentate.

16. The composition of claim 15 wherein the one or more bacteria is selected from the group consisting of Clostridium sporogenes, Peptostreptococcus anaerobius, Clostridium cadaveris, Clostridium boltae, and combinations thereof.

17. The composition of claim 15 wherein the fermentate further comprises one or more proteolytic enzymes.

18. The composition of claim 17 wherein the fermentate further comprises additional nutrients selected from the group consisting of carbohydrates, soy flour, and combinations thereof.

19. The composition of claim 15 wherein the composition comprises from about 0.03% to about 50%, by weight of the composition, of the fermentate.

20. A method of promoting brain health comprising administering to an individual in need thereof the composition of claim 15.


SEQUENCE LISTING (1 sequence)

SEQ ID NO: 1 - DNA, 4896 bp, Clostridium sporogenes <s300> Dickert, S., Pierik, A.J. and Buckel, W. Molecular characterization of (r)-phenyllactate dehydratase and its activating component A from Clostridium sporogenes Laboratorium fuer Mikrobiologie 44 1 49-60 2002-04-01 AF420489 2016-07-26
aattccctttaacagatacaggtaaaattaagaggcatgaactaaaaaaatgctttgaaaagaagtttgaattaagacaatctatttaaattaataataaatatattaaattaacaataaatatattaaattaacaataaatctatttaaggaggctttttttatggaaaacaatacaaatatgtttagtggagtaaaggttattgaattagcaaattttatagctgctccagcagcaggtagattttttgctgatggtggtgcagaggtaataaaaattgaatcacctgctggagatcctttaagatatactgctccttcagaaggaagaccattaagccaagaagaaaatactacttatgatttggaaaatgcaaataaaaaagcaatagtattaaatcttaaaagcgaaaaaggtaaaaagatattacatgaaatgttagcagaagcagatatattattaactaattggagaacaaaggctttagttaaacaaggattagactatgaaacactaaaagaaaaatatcctaaattagtttttgcacaaataactggttatggtgaaaaaggaccagataaagatcttccaggctttgattatactgcatttttcgctagaggcggtgtttcaggtactctttatgaaaaaggaactgtgcctccaaatgttgttccaggacttggagaccatcaagctgggatgtttttagcagcgggtatggcaggagctttatataaagcaaaaacaacaggacaaggagataaagtaacagtaagtttaatgcatagtgctatgtatggactaggtattatgatacaagctgctcaatataaagatcatggattagtatatccgataaatcgtaatgaaactccaaatccttttatagtttcatataaatctaaggatgattactttgttcaagtatgtatgccaccatatgatgttttctatgatagatttatgaccgctttaggaagagaagatttagttggagacgaaagatacaataaaatagaaaatttaaaagatggacgtgctaaggaagtatacagtataatcgaacaacaaatggttacaaagacaaaggatgaatgggataacatatttagagatgcagacattccatttgctatcgcacaaacttgggaagatttattagaagatgaacaagcttgggcaaatgattatttgtataagatgaaatatccaacaggaaacgaaagagcattagtaagacttccagtattctttaaagaagcaggattaccagaatataatcaatcaccacaaatagcagaaaatactgtagaagttttaaaagaaatgggatatacagaacaagagattgaggaattagaaaaagataaagatataatggtaaggaaggaaaaataatggcagacatttatactatgggtgtagacataggttcaactgcatcaaaaacagtagtattaaaaaatggtaaagaaattgtaagtcaagcagtaataagtgtaggggccggaacaagtggccccaagagagctatagattctgtattaaaagatgctaaattatccattgaagatttagactatattgtatccactggatatggaagaaatagtttcgattttgctaacaaacaaatttctgaattaagttgtcatgcaaaaggggtctatttcgataacaataaagctagaacagttattgatataggcggacaagatattaaagtattaaaattagcggatagtggaagacttttaaactttataatgaatgataaatgtgctgcaggaacgggacgatttttagatgtaatgtctagagtaatagaagttccagttgatgagttaggaaaaaaagcattagaaagcaaaaatccttgtactattagttctacctgtacagtatttgcagagtcagaagtaatttctcaacttgcaagaggagttaaaactgaagatttgatagcaggaatttgtaaatctgtagcatcaagagtggctagccttgcaaagagaagtggtatagaagaattagtagttatgagtggaggagtagctaaaaatataggtgtagtaaaggcaatggaagcagaattgggaagagacatatatatatctaaaaattctcaattaaatggagcattgggagcaagtctatacgcttatgaaagttttcaaaaagaaaggagctaaaaacatgagtgatagaaataaggaagtaaaagaaaaaaaggcaaagcattatcttagagagattactgcaaagcattacaaagaagctctcgaagcaaaagaaaggggagaaaaggttggttggtgtgcatctaacttcccacaagaaatagctacaacattgggggtaaaagttgtttatccagaaaatcatgcagcagctgtagcagctagagggaatggacaaaatatgtgtgaacatgctgaggctatgggtttttctaatgatgtatgtggttatgcaagagtaaatttagctgttatggacataggtcatagtgaagatcaaccaatacctatgccagactttgtactttgctgtaataacatttgtaatcaaatgattaaatggtatgagcatatagcaaaaactttagatataccaatgattcttatagatataccatacaatacagaaaatactgtttcacaagatagaattaaatatattagagcacaatttgatgatgcaataaaacaattggaagaaataacaggcaaaaaatgggatgaaaataaatttgaagaagttatgaaaatatcccaagaaagtgcaaaacaatggttaagagcagcatcctatgcaaagtataaaccttcaccatttagcggatttgatttatttaatcatatggctgtagcagtttgtgcaagaggtacacaagaagctgcagatgcatttaagatgttagcagatgaatatgaggagaatgtaaaaactggaaaatccacttataggggagaagaaaaacaacgtatattatttgaagggattgcctgttggccatatttgagacataaattaactaagcttagtgaatatggtatgaacgtaactgcaactgtatacgcagaagcctttggtgttatatatgagaatatggatgaattaatggctgcttataataaagttcctaattcaattagttttgaaaacgcattaaaaatgagattaaatgctgttacaagcactaatacagaaggtgctgttattcatataaatagaagctgtaaattatggagtggatttttatatgagctagcaagaagattagaaaaggaaacaggaattcctgtagtatcatttgatggggaccaggcagacccaagaaatttctcagaagctcaatatgatactagaattcaaggacttaatgaagtaatggttgctaaaaaggaggctgaataagatgtcaaattcagataaattttttaatgactttaaggatattgtagaaaatcctaaaaaatatataatgaagcatatggaacaaactggacaaaaggctataggatgtatgccattatatactcctgaggaacttgtattagctgctggaatgtttccagtaggggtatggggaagcaatacagaactttcaaaagctaaaacatatttcccagcatttatttgttcaatattacaaacaacattggaaaatgcattaaatggagaatatgatatgttatctggtatgatgattacaaattattgtgattcattaaaatgcatgggacaaaattttaaactaaccgttgaaaatattgagtttatcccagtaacagttccacaaaatagaaaaatggaagctggaaaagagtttttaaaaagtcaatataaaatgaatattgagcaattagaaaagatttctggtaataaaataacagatgaatctttagaaaaagctatagaaatatatgatgaacacagaaaagtaatgaatgacttttcaatgttagcatcaaaatatccaggtataataacaccaactaaacgtaattatgttatgaaatctgcttattatatggataaaaaagaacatactgaaaaagttagacaattaatggatgaaattaaagctatagaaccaaaaccatttgaaggaaagagagttataactacaggtataattgcagattcagaagatttacttaaaatattagaagaaaataatatagctatagttggtgatgatatagcacatgaatctagacaatatagaacattgactccagaagcgaacacaccaatggataggttagctgagcaatttgctaatagagaatgtagtactttatatgatcctgaaaagaaaaggggtcaatatatagtagaaatggctaaagagagaaaagcagatggaattatatttttcatgacaaaattctgtgacccagaggaatatgattatccacaaatgaaaaaggattttgaagaagcaggcattccacatgtactaatagaaactgatatgcaaatgaaaaattatgaacaagctagaactgcaattcaggctttttcagaaacactttaataaaagttttcaatatttactgtaaactttattaattgaaacattgatttctcttctctttctataaaataatatttatatttaaaaaagttatgtttagatggatgaaaggaaatcaatgttcatataaattaacaaattcattaatatattaggagggatataatgtttttcacagaacaacatgaacttattagaaaattagcaagagattttgcagagcaggaaatagagcctattgcagatgaagtagataaaactgccgagttccctaaagaaattgtgaaaaaaatggcccaaaatggtttttttggaataaaaatgcctaaagaatatggtggagctg